(19) United States
(12) Patent Application Publication
(10) Pub. No.: US 2023/0076688 A1
(43) Pub. Date: Mar. 9, 2023

(54) METHOD FOR PREPARING INDUCED PLURIPOTENT STEM CELL LINE FROM MESENCHYMAL STEM CELLS, AND CELL LINE OBTAINED THEREBY

(71) Applicant: BBHC CO., LTD., Seoul (KR)

(72) Inventors: Sang Yeon LEE, Gyeonggi-do (KR); Won Ju JUNG, Seoul (KR); Ho Bin KIM, Seoul (KR); Min Sun OH, Seoul (KR); Kye Ho LEE, Seoul (KR)

(21) Appl. No.: 17/929,364

(22) Filed: Sep. 2, 2022

Related U.S. Application Data
(62) Division of application No. 15/328,656, filed on Jan. 24, 2017, now abandoned.

(30) Foreign Application Priority Data
Jul. 25, 2014 (KR) 10-2014-0094601

(51) Int. Cl. C12N 5/074; A61K 35/545; A61K 35/30; A61K 35/32; A61K 35/407

(52) U.S. Cl. CPC C12N 5/0696; A61K 35/545; A61K 35/30; A61K 35/32; A61K 35/407; C12N 2500/76; C12N 2506/1369; C12N 2506/1392

(57) ABSTRACT

The present invention relates to a method for preparing an induced pluripotent stem cell line from mesenchymal stem cells; and an induced pluripotent stem cell line (deposit number: KCLRF-BP-00318) obtained thereby. Specifically, the method for preparing an induced pluripotent stem cell line, of the present invention, comprises the steps of: (a) obtaining mesenchymal stem cells from a human umbilical cord; (b) forming, from the mesenchymal stem cells, a colony with a medium for dedifferentiation containing an Ecklonia cava extract; and (c) obtaining an induced pluripotent stem cell line by sub-culturing the colony. The induced pluripotent stem cell line according to the present invention was first established by the present inventors, and the pluripotent stem cell line of the present invention can be differentiated into various cells and can treat various diseases or disorders through cell transplant therapy.

This application is a continuation of application Ser. No. 15/328,656, filed on Jan. 24, 2017, which is a national stage application of International Patent Application No. PCT/KR2014/007207, filed Aug. 5, 2014, which claims priority to Korean Patent Application No. 10-2014-0094601, filed Jul. 25, 2014. The entirety of the aforementioned applications are incorporated herein by reference.

### FIELD

The present invention relates to a method for preparing an induced pluripotent stem cell line from mesenchymal stem cells; and a pluripotent stem cell line obtained thereby.

### BACKGROUND

A cell line means a serially passed cell system, that is, an established cell line and means that cultured cells acquire infinitely proliferation to become a serially passed cell system.

Further, stem cells are collectively referred to as undifferentiated cells before differentiation that can be obtained from each tissue. The stem cells have a property capable of continuously making the same cells as itself for a predetermined period in an undifferentiated state and a property capable of being differentiated into various cells configuring a biological tissue under a proper condition.

The stem cells may be largely classified into embryonic stem cells and adult stem cells depending on differentiation potency and a creation time. As another classification, the stem cells may be divided into pluripotent, multipotent, and unipotent stem cells depending on differentiation potency of the stem cells.

The adult stem cells may be classified into multipotent or unipotent stem cells. Representative adult stem cells include mesenchymal stem cells (MSCs) and hematopoietic stem cells (HSCs). The MSCs are differentiated into chondroblast, osteoblast, adipocyte, myocyte, and neuron, and the HSCs are differentiated into blood cells in the blood including red blood cells, white blood cells, platelets, and the like.

On the other hand, the pluripotent stem cells are called stem cells having multifunctions which may be differentiated into three germ layers configuring a living body to be differentiated into all cells or organ tissues of the human body and generally, the embryonic stem cells correspond to the pluripotent stem cells. It is known that the human embryonic stem cells are made from the embryos which may be generated from the human organism to have many ethical issues, but have excellent cell proliferation and differentiation potency as compared with the adult stem cells. The adult stem cells may be obtained from bone marrow, blood, brain, skin, etc. to have less ethical issues, but have limited differentiation potency as compared with the embryonic stem cells.

As an alternative to overcome the problems, various methods for manufacturing customized pluripotent stem cells (cell line) similar to the embryonic stem cells by dedifferentiating cells derived from the adult have been attempted. As a representative method, there are a fusion with ES cell method, a somatic cell nuclear transfer method, a reprogramming by gene factor method, and the like. The fusion with ES cell method has a problem in terms of cell stability because the induced cells further have two pairs of genes, and the somatic cell nuclear transfer method has a problem in that a lot of ova are required and efficiency is too low. In addition, the reprogramming by gene factor method is a method using virus containing oncogenes in order to induce dedifferentiation by inserting a specific gene and has a problem in terms of development of cell therapeutic agents due to a high risk of cancer occurrence, low efficiency, and difficulty in a methodical aspect.

In order to successfully obtain a large amount of pluripotent stem cells, a culture composition is very important in the step of culturing isolated adipose-derived monocytes, and thus, researches for manufacturing a larger amount of pluripotent stem cells by an induction method with high efficiency are required.

Meanwhile, in some cases, Ecklonia cava is used for a composition for treating or preventing an atopic disease (Korean Patent Application Publication No. 2009-0043115) or a hair dye composition for oxidation dyeing (Korean Patent Application Publication No. 2012-0126148), but has been never used for dedifferentiating adipose-derived mesenchymal stem cells into an induced pluripotent stem cell line.

Details described in the above background are only for enhancement of understanding of the background of the present invention and therefore it may contain information that does not form the prior art that is already known in this country to a person of ordinary skill in the art.

### BRIEF DESCRIPTION OF THE DRAWINGS

FIG. 1 is a diagram illustrating that substantially the same pluripotent stem cells as embryonic stem cells are induced in mesenchymal stem cells by injecting a dedifferentiation medium (STC-F002) containing an Ecklonia cava extract to a mesenchymal stem cell and then culturing the medium.

FIG. 2 illustrates formation of colonies of induced pluripotent stem cells according to a concentration of an Ecklonia cava extract by a method (Example 3) of the present invention.

FIG. 3 verifies that cells (Experimental Example 1) induced by the method of the present invention are pluripotent stem cells by using expression of SSEA-4 which is a pluripotent stem cell-specific protein.

FIG. 4 verifies that cells (Experimental Example 2) induced by the method of the present invention are pluripotent stem cells by using expression of a pluripotent stem cell-specific protein.

FIG. 5 illustrates gene expression (Experimental Example 3) of the pluripotent stem cells induced by the method of the present invention.

FIGS. 6 to 8 verify the pluripotent stem cells by inducing differentiation of the pluripotent stem cells induced by the method of the present invention into ectodermal cells, mesodermal cells, and endodermal cells.

### DETAILED DESCRIPTION

### Technical Problem

The inventors made an effort to find a method for inducing a pluripotent stem cell line with high efficiency for application of developing cell therapeutic agents having high safety and high production efficiency. As a result, the inventors verified that when an Ecklonia cava extract as a safe natural extract is added to a cell culture medium, an induced pluripotent stem cell line can be prepared with safe and high efficiency by using mesenchymal stem cells. Accordingly, the inventors completed the present invention.

Therefore, an object of the present invention is to provide a method for preparing an induced pluripotent stem cell line by adding a dedifferentiation medium (hereinafter, referred to as STC-F002) containing an Ecklonia cava extract in mesenchymal stem cells.

Another object of the present invention is to provide an induced pluripotent stem cell line EPN-1 (deposit number: KCLRF-BP-00318) dedifferentiated by culturing mesenchymal stem cells in a dedifferentiation medium containing an Ecklonia cava extract.

Yet another object of the present invention is to provide a composition for cell therapy including the induced pluripotent stem cell line.

Other objects and advantages of the present invention will be more apparent by the detailed description of the invention, claims, and drawings below.

### Technical Solution

One aspect of the present invention provides a method for preparing an induced pluripotent stem cell line from mesenchymal stem cells, in which the method includes the steps of: (a) obtaining mesenchymal stem cells from a human umbilical cord; (b) forming, from the mesenchymal stem cells, a colony with a dedifferentiation medium containing an Ecklonia cava extract; and (c) obtaining an induced pluripotent stem cell line by sub-culturing the colony.

The inventors made an effort to find a method for inducing a pluripotent stem cell line with high efficiency for application of developing cell therapeutic agents having high safety and high production efficiency without ethical issues to destroy the embryo. As a result, it is verified that when the Ecklonia cava extract as a safe natural extract is added in the cell culture medium, remarkably, the pluripotent stem cell line can be manufactured with high efficiency.

Ecklonia cava which is an active ingredient included in the medium composition for dedifferentiation of the present invention is a perennial alga of a laminariaceous laminariales brown plant that mainly lives the southern coast, the coast of Jeju Island, and the coast of Ulleungdo Island, mainly is food for abalone, turban, and the like, and is used as a main raw material to make alginate or potassium iodide or for food.

The Ecklonia cava extract included in the present invention may be extracted by using water and organic solvents including (a) anhydrous or water-containing low alcohol having 1 to 4 carbons (methanol, ethanol, propanol, butanol, n-propanol, iso-propanol, n-butanol, etc.), (b) a mixed solvent of the low alcohol and water, (c) acetone, (d) ethyl acetate, (e) chloroform, (f) 1,3-butylene glycol, (g) hexane, (h) diethyl ether, and the like, and preferably, may be extracted by using a mixed solvent of methanol or ethanol and water. In the case of extracting the Ecklonia cava extract by using the mixed solvent, the content of methanol or ethanol may be 50 to 80 v/v %.

Currently, cases for applying the Ecklonia cava extract to skin compositions such as cosmetics have been increased (see Korean Patent Application Publication Nos. 2013-0017159, 2012-0040488, and 2010-0097293, etc.), but there is no case for developing the Ecklonia cava extract into a pluripotent stem cell-induced media.

The term “embryonic stem cells” used in the present invention are called cells having pluripotency as cells which are isolated and cultured from an inner cell mass of blastocyst in the early days of its development after fertilization. The term “pluripotent stem cells” used in the present invention are called stem cells having pluripotency which may be differentiated into three germ layers configuring the living body, that is, an endoderm, a mesoderm, and an ectoderm.

The term “differentiation” used in the present invention means that while the cells are divided, proliferated, and grown, structures or functions thereof are specialized, that is, forms or functions are changed in order to perform tasks which are given to cells, tissues, and the like of an organism.

The term “cell therapeutic agent” of the present invention, as a drug used for treating, diagnosing, and preventing by using cells and tissues manufactured through isolation from the human, culture, and a specific manipulation, is referred to as a drug used for treating, diagnosing, and preventing through a series of actions such as proliferating and screening homogenous or heterogeneous cells for restoring functions of cells or tissues, changing a biological characteristic of the cells by another method, and the like. The cell therapeutic agents are largely classified into somatic cell therapeutic agents and stem cell therapeutic agents according to differentiation of cells, and the present invention relates to stem cell therapeutic agents.

The “mesenchymal stem cells” of the present invention are cells isolated from embryonic stem cells or adult stem cells derived from mammals, preferably umbilical cord-derived mesenchymal stem cells, and more preferably human umbilical cord-derived mesenchymal stem cells. The stem cells may be extracted and obtained from the umbilical cord connecting placenta and fetus in human body. The extraction of the mesenchymal stem cells from the umbilical cord may be performed by using various methods, and for example, the umbilical cord is extracted from the human body and washed with a DPBS until the blood does not flow, and the washed umbilical cord is chopped with a surgical blade and cultured at 37° C. to obtain a solution containing monocytes.

The term “medium” used in the present invention means a mixture for culturing or differentiating cells such as stem cells in vitro, which contains required elements for growth and proliferation of the cell including sugars, amino acids, various nutrients, serum, growth factors, minerals, and the like.

Various media are commercialized in the art and may be artificially manufactured and used. For example, as the commercialized medium, a Dulbecco's modified eagle's medium (DMEM), a minimal essential medium (MEM), a basal medium eagle (BME), RPMI 1640, F-10, F-12, DMEM F-12, a a-minimal essential medium a-MEM), a Glasgow's minimal essential medium (G-MEM), an Iscove's modified Dulbecco's medium (IMPM), AmnioMax, an AminoMax II complete medium (Gibco, N.Y., USA), and a Chang's medium MesemCult-XF medium (STEMCELL Technologies, Vancouver, Canada), and the like are included, and may be used as a basic medium included in the medium composition of the present invention in addition to a medium which may be artificially manufactured.

In the basic medium, generally added serum ingredients (for example, fetal bovine serum (FBS)), antibiotics (for example, penicillin and streptomycin), and the like may be added. The concentration of the serum ingredient or the antibiotic ingredient which is added in the basic medium may be modified within a range that can achieve the effect of the present invention, and preferably, 10% FBS, 100 unit/ml of penicillin, 50 μg/ml of streptomycin, and the like, may be added.

Further, the medium of the present invention may additionally include a nutrient mixture. The nutrient mixture is a mixture containing various amino acids, vitamins, inorganic salts, and the like which are generally used in a cell culture and may use a nutrient mixture which is manufactured by mixing the amino acids, the vitamins, the inorganic salts, and the like or commercially manufactured. The commercially manufactured nutrient mixture may include M199, MCDB110, MCDB202, MCDB302, and the like as an example, but is not limited thereto.

Further, the medium of the present invention may additionally include energy water for induction and stabilization of the pluripotent stem cells. The energy water is preferably added in the amount of 0.01 to 0.01 v/v % and more preferably 0.05 to 0.5 v/v %.

The medium composition of the present invention is a pluripotent stem cell-induced specific medium and may be achieved by adding the Ecklonia cava extract to the basic medium, and may include the Ecklonia cava extract at a concentration of preferably 1 to 1,000 μ/ml and more preferably 1 to 400 μg/ml based on the entire medium composition.

The ‘induced pluripotent stem cell line’ of the present invention means a continuously sub-culturable cell line as stem cells inducing pluripotency such as embryonic stem cells from mesenchymal stem cells having multipotency. For the purpose of the present invention, the induced pluripotent stem cell line means preferably EPN-1 (deposit number: KCLRF-BP-00318).

Another aspect of the present invention provides an induced pluripotent stem cell line EPN-1 (deposit number: KCLRF-BP-00318) dedifferentiated by culturing mesenchymal stem cells in a dedifferentiation medium containing an Ecklonia cava extract.

The an induced pluripotent stem cell line EPN-1 of the present invention was deposited as a deposit number of KCLRF-BP-00318 on May 30, 2014 at the Korean Cell Line Research Foundation, College of Medicine, Seoul National University.

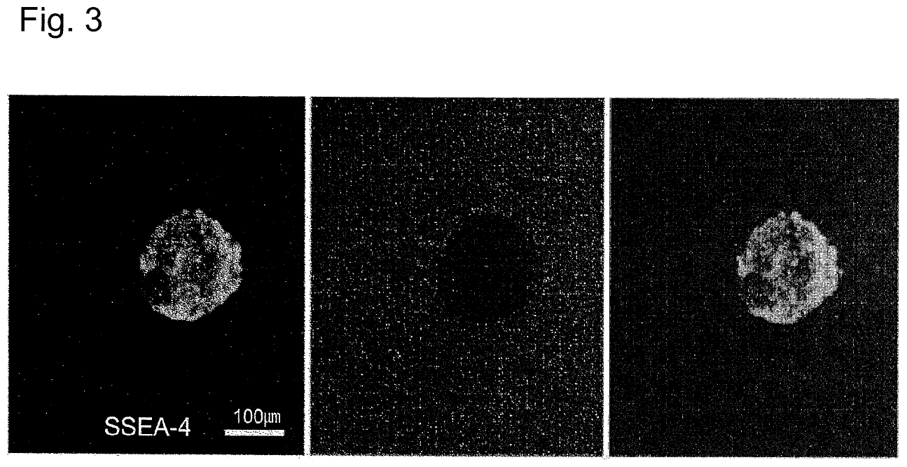

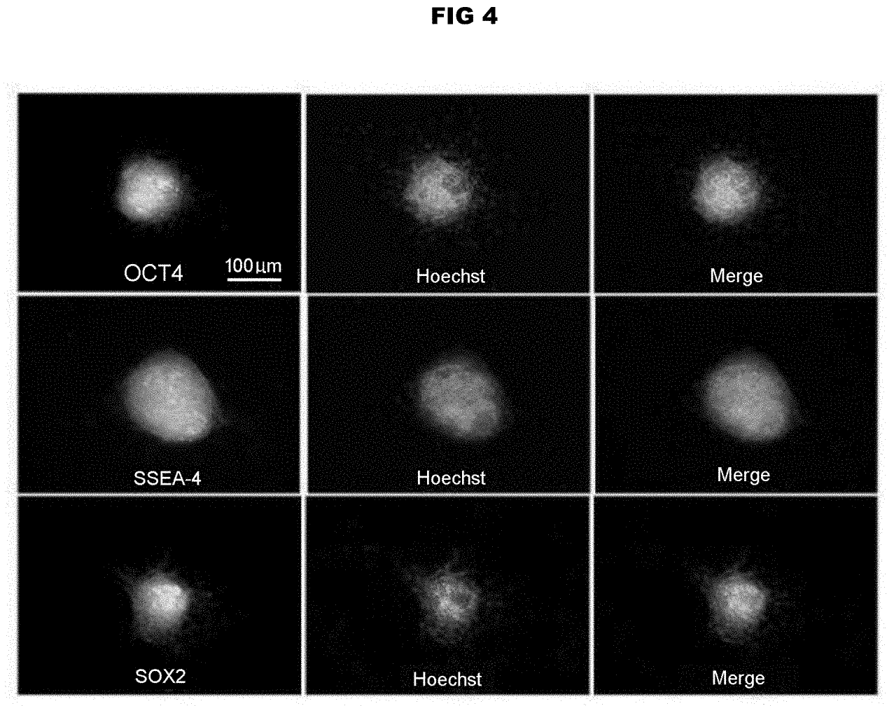

Preferably, provided is the induced pluripotent stem cell line EPN-1 characterized by showing a positive response in a straining reaction for Oct-4, SOX-2, or stage-specific embryonic antigen-4 (SSEA-4). In an exemplary embodiment of the present invention, it was proved that this was the pluripotent stem cell line by testing characteristics of the induced pluripotent stem cell line (FIGS. 3 and 4).

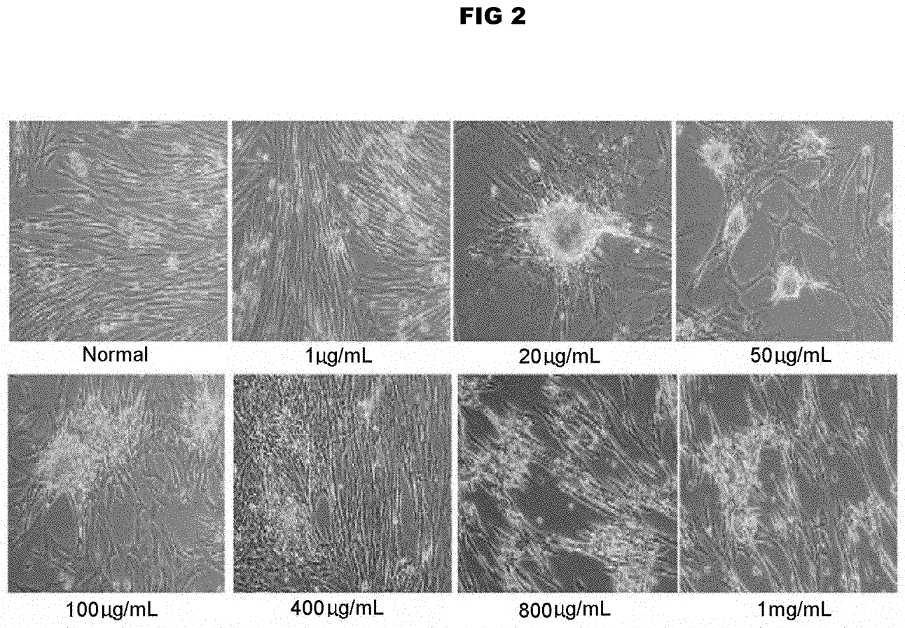

According to the exemplary embodiment of the present invention, in the case of using the medium composition containing the Ecklonia cava extract of the present invention, unlike the case of using only a DMEM F-12 medium, it was verified that pluripotent stem cell colonies were formed at 8 to 10-th days (FIG. 2).

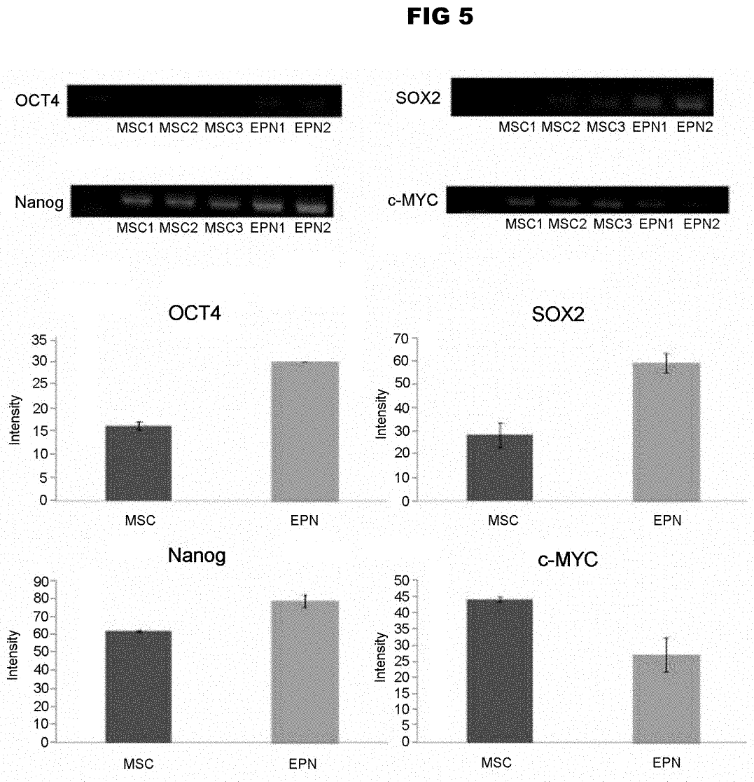

The induced pluripotent stem cell line of the present invention has the same potency as the embryonic stem cells and almost the same as the embryonic stem cells in shapes of the cells. According to an exemplary embodiment of the present invention, as a result of examining whether to express specific genes, Nanog, Oct4, Sox-2, and c-Myc and a protein SSEA-4 in the embryonic stem cells, it is verified that the genes and the protein are expressed in the pluripotent stem cells induced by the present invention like the embryonic stem cells (see FIGS. 4 and 5).

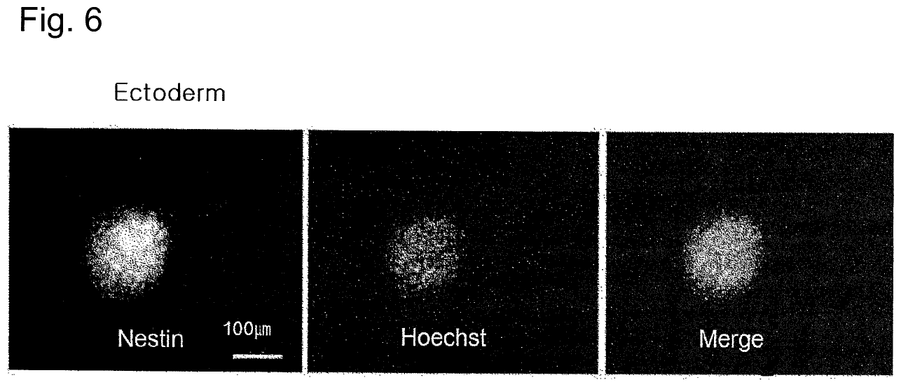

Further, the induced pluripotent stem cell line of the present invention is differentiated into nerve cells that are ectoderm cells, hepatocytes that are endoderm cells, and cartilage and osteoblasts that are mesodermal cells and has the same potency as the embryonic stem cells and has the same differentiation potency as the embryonic stem cells by verifying that the induced pluripotent stem cell line are differentiated into ectoderm, endoderm, and mesoderm like the pluripotent stem cells by verifying differentiation through each specific straining reaction (nerve cells (Nestin), hepatocytes (.alpha.-fetrotein), cartilage (Alcian blue), and osteoblasts (Von kossa)) (see FIGS. 6 to 8).

Accordingly, the induced pluripotent stem cell line of the present invention may be used as an effective cell therapeutic agent.

The composition of the present invention may be administrated by any administration route, particularly, a method such as peritoneal or thoracic cavity administration, subcutaneous administration, intravenous or endovascular administration, intramuscular administration, local administration by injection, or the like.

In the present invention, the composition may be administrated in a form such as Injections, suspensions, and emulsions on the basis of a general method, and if necessary, may be suspended in an adjuvant such as a Freund's complete adjuvant or administrated together with a material having an adjuvant activity such as BCG.

The cell therapeutic composition of the present invention can be applied to arthritis, neurological disorders, endocrine disorders, liver diseases, and the like and has a possibility to an allergenic therapeutic agent for the human according to clinical trial results for the human later.

### Advantageous Effects

Features and advantages of the present invention are as follows.

(i) The present invention provides a method for preparing an induced pluripotent stem cell line from mesenchymal stem cells by using a dedifferentiation medium containing an Ecklonia cava extract.

(ii) The present invention provides an induced pluripotent stem cell line EPN-1 (deposit number: KCLRF-BP-00318) cultured and dedifferentiated in a dedifferentiation medium containing an Ecklonia cava extract and the induced pluripotent stem cell line is first established by the inventors.

(iii) The present invention provides a cell therapeutic composition including an induced pluripotent stem cell line EPN-1 (deposit number: KCLRF-BP-00318).

(iv) When the medium composition according to the present invention is used, the induced pluripotent stem cell line can be efficiently prepared using the mesenchymal stem cells. In addition, since the prepared induced pluripotent stem cell line can be differentiated into various cells, the induced pluripotent stem cell line can be usefully used as a cell therapeutic agent.

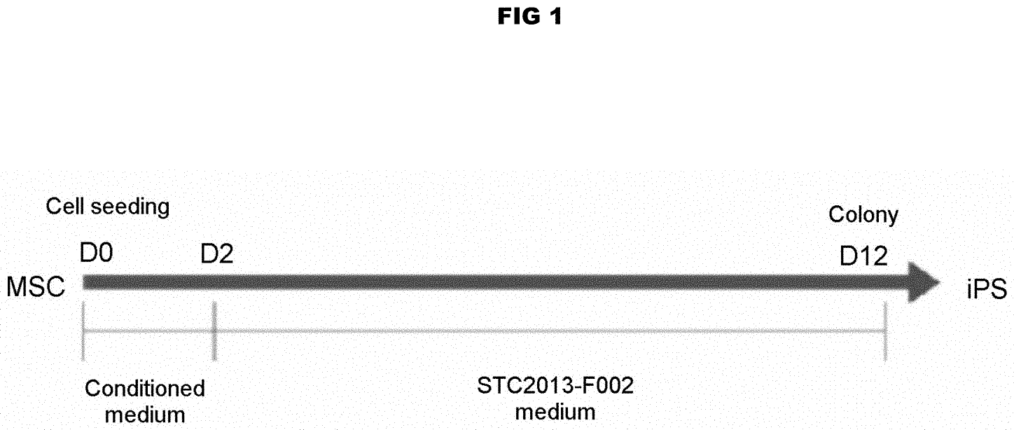

FIG. 1 is a diagram illustrating that substantially the same pluripotent stem cells as embryonic stem cells are induced in mesenchymal stem cells by injecting a dedifferentiation medium (STC-F002) containing an Ecklonia cava extract to a mesenchymal stem cell and then culturing the medium.

FIG. 2 illustrates formation of colonies of induced pluripotent stem cells according to a concentration of an Ecklonia cava extract by a method (Example 3) of the present invention.

FIG. 3 verifies that cells (Experimental Example 1) induced by the method of the present invention are pluripotent stem cells by using expression of SSEA-4 which is a pluripotent stem cell-specific protein.

FIG. 4 verifies that cells (Experimental Example 2) induced by the method of the present invention are pluripotent stem cells by using expression of a pluripotent stem cell-specific protein.

FIG. 5 illustrates gene expression (Experimental Example 3) of the pluripotent stem cells induced by the method of the present invention.

FIGS. 6 to 8 verify the pluripotent stem cells by inducing differentiation of the pluripotent stem cells induced by the method of the present invention into ectodermal cells, mesodermal cells, and endodermal cells.

### Modes of the Invention

Hereinafter, the present invention will be described in more detail through Examples. However, the present invention is not limited to the exemplary embodiments disclosed below, but can be implemented in various forms. The following exemplary embodiments are described in order to enable those of ordinary skill in the art to embody and practice the invention.

### EXAMPLES

### Example 1: Preparation of Dedifferentiation Medium (Hereinafter, Referred to as ‘STC-F002’)

Herb medicine samples used in an experiment were purchased in Jeju Island, exactly evaluated by an expert, and used in the experiment. 100 g of a dried herb medicine sample was added in 1 L of water, and then, the obtained water was extracted for 16 hours by applying an ultrasonic extractor, and filtrated by using a filter. A filtrate was concentrated in a rotary decompression evaporator and immediately lyophilized. 1.about.1000 μg/ml of a Jeju Ecklonia cava extract and 0.1 v/v % of energy water were mixed to prepare a STC-F002 medium as a dedifferentiation medium.

### Example 2: Isolation and Culture of Mesenchymal Stem Cells from Human Umbilical Cord

### Example 2-1: Collection of Human Umbilical Cord

An umbilical cord tissue was collected immediately after birth. A sample was first clearly rinsed before being transferred to a laboratory and then immediately transferred to 500 ml of a sterile glass bottle containing a F-12 medium added with a transfer medium (50 IU/ml of penicillin and 50 μg/ml of streptomycin (purchased from Invitrogen)). In the laboratory, stem cells were extracted in a flow hood of class 100 under a sterile condition. The sample was first transferred to a sterile stainless steel container. The sample was washed with PBS several times and then the umbilical cord tissue sample was cut with a length of 2 cm and transferred to a cell culture dish having a diameter of 10 cm, and herein, additionally washed and treated with 70% ethanol for anti-infection, and then washed several times with PBS added with an antibiotic mixture (50 IU/ml of penicillin and 50 μg/ml of streptomycin (purchased from Invitrogen)) until the solution was cleaned.

### Example 2-2: Isolation and Culture of Stem Cells from Human Umbilical Cord

In order to isolate Warton jelly (a substance of the umbilical cord) from the blood vessel and other internal components of the umbilical cord, cutting of the umbilical cord tissue was first performed. After removing the blood vessel, the isolated Warton jelly was cut with sizes of small pieces (0.5 cm×0.5 cm) in order to extract the cells. The explant was performed by putting the Warton jelly pieces of the umbilical cord in respective tissue culture dishes under a cell culture condition suitable for the extraction of epithelial stem cells or the mesenchymal stem cells.

For isolation/culture of the mesenchymal stem cells, the explanted tissue was immersed in 5 ml of a Dulbecco's modified eagle medium (DMEM) F-12 (Gibco) added with 10% fetal bovine serum (FBS, Hyclone), 10% FBS, 100 unit/ml of penicillin, and 50 μg/ml of streptomycin and maintained at 37° C. in a carbon dioxide cell incubator. The medium was replaced every 3 or 4 days. The outgrowth of the cells was monitored by an optical microscope. The outgrown cells were treated with Trypsin (0.125% Trypsin/0.05% EDTA) for additional expansion and refrigeration (using DMEM/10% FBS).

The medium was replaced every 3 or 4 days. The outgrowth of the cells from the explanted tissue was monitored by an optical microscope.

For extraction of the mesenchymal stem cells, pellets of the cells were re-suspended and counted in the medium DMEM F-12 (Gibco), 10% FBS, 100 unit/ml of penicillin, and 50 μg/ml of streptomycin and inoculated on a tissue culture dish of 10 cm at a density of 1×10⁶ cells/dish. The medium was replaced every 3 or 4 days. The growth and clone formation of the cells were monitored by an optical microscope. In approximately 90% cell number (confluence), the cells were sub-cultured as described above.

### Example 3: Preparation of Pluripotent Stem Cells from Human-Derived Mesenchymal Stem Cells According to Concentration of Ecklonia cava Extract in Dedifferentiation Medium

As an experiment for inducing pluripotent stem cells from human umbilical cord-derived stem cells according to a concentration of a STC-F002, in a control group, DMEM F-12 (Gibco) as a dedicated medium of MSC, 10% FBS, 100 unit/ml of penicillin, and 50 μg/ml of streptomycin were used as a basic medium, and in an experimental group, human umbilical cord-mesenchymal stem cells which were sub-cultured twice were used, and in the medium, the Jeju Ecklonia cava extract at concentrations of 1 μg/ml, 20 μg/ml, 50 μg/ml, 100 μg/ml, 400 μg/ml, 800 μg/ml, and 1,000 μg/ml and 0.1 v/v % of energy water were added (see FIG. 2). The human umbilical cord-derived mesenchymal stem cells were isolated and the washed monocytes were inoculated in a 6-well plate (dish) in the amount of 1×10⁴ cells and maintained and cultured at 37° C. and 5% CO2. As the cultured result, it was verified that in the medium containing 1 to 400 μg/ml of the Ecklonia cava extract, the colonies were formed.

### Example 4: Establishment of Stem Cell Line by Sub-Culturing Colonies

The colonies generated in Example 3 were treated with 1 mg/ml of collagenase to isolate colony cells and the isolated cells were inoculated in a T175 flask in the cell number of 1×10⁶ in a DMEM/F12 medium containing 10% FBS, 100 unit/ml penicillin, and 50 μg/ml of streptomycin to be cultured in a CO2 incubator, the medium was replaced every 2 to 3 days, and sub-cultured twice under a condition of performing sub-culture at confluency 80% to establish a stem cell line.

### Experimental Example 1: Check Whether or not Induced Pluripotent Stem Cell Line

Whether the cell line cultured by the method of Example 4 had features as the pluripotent stem cell line was verified by the following method.

Particularly, it was verified that the stem cells sub-cultured by the method of Example 4 continuously formed the colonies, and as an analyzed result of a confocal microscope by performing immunochemical straining using a SSEA-4 antibody as a specific marker of the pluripotent stem cells, it was verified that since only the colony cells were strained with the marker, only the cells in the colony were the pluripotent stem cells (FIG. 3). Further, it was verified that even through sub-culture for 6 months, the stem cells were continuously proliferated to be the cell line.

Accordingly, the inventors named the cell line as “EPN-1 cell” and deposited the cell line as a deposit number KCLRF-BP-00318 in the Korean Cell Line Research Foundation (Cancer Research Institute, College of Medicine, Seoul National University, 28, Yeongeon-dong, Jongno-gu, Seoul, Korea) at May 30, 2014.

### Experimental Example 2: Analysis Whether to Express Protein of Pluripotent Stem Cells

With respect to the pluripotent stem cells prepared in Example 3, whether to express OCT4, SOX2, and stage-specific embryonic antigen4 (SSEA4) as specific proteins of the embryonic stem cells was analyzed by using an immunochemical staining method using antibodies thereto. In the staining process, cells were first fixed by using 4% paraformaldehyde and washed with PBS, and blocked with a 1% BSA solution.

The cells were treated with primary antibodies for OCT4, SOX2, and SSEA-4 and reacted at 4° C. for 18 hours, and then washed with PBS, treated with secondary antibodies with fluorescein isothiocyanate (FITC) to the primary antibodies, and reacted at room temperature for 1 hour. Thereafter, in order to strain DNAs of the cells, a hochest dye was used, and as a result, the nuclei of the cells were strained. The cells were washed with PBS and then the expression was analyzed by using a fluorescence microscope, and the result thereof was illustrated in FIG. 3.

The protein straining was photographed at a wavelength of 488 nm by using the FITC and the hochest was photographed at a wavelength of 350 nm as a UV wavelength and then was not overlapped with the FITC wavelength. The first diagram means a straining result for each protein expression and the gene expression in the nuclei and the hochest means that the nuclei of the cells were strained by using a hochest dye, and the third diagram illustrates a combination of the two diagrams (FIG. 4).

As a result, in the experimental group, only when the concentration of the Jeju Ecklonia cava extract was 1 to 400 μg/ml, it was observed that the colonies were formed after 10 days (see FIG. 2) and only the colonies were strained with OCT4, SOX2, and SSEA-4 as the pluripotent stem cell-specific markers and verified as the pluripotent stem cells (FIG. 4).

### Experimental Example 3: Comparison of Genetic Analysis of Pluripotent Stem Cells

While the pluripotent stem cells prepared in Example 3 were observed by a microscope, only the colonies were picked by using a pipette of 200 μl, and then the total RNA was isolated by using a TRIzol reagent (manufactured by Invitrogen Corporation). cDNA was synthesized by using reverse transcription-polymerase chain reaction (RT-PCR) and then the PCR was performed by using specific primers to OCT4, Sox-2, Nanog, c-Myc, and a glyceraldehyde 3-phosphate dehydrogenase (GAPDH) gene as a control gene.

The Nanog, OCT4, and Sox-2 are specific genes in the embryonic stem cells, and the c-Myc gene is a non-specific gene which may be positive in both the embryonic stem cells and the adult stem cells. The PCR product was analyzed by agarose gel electrophoresis and a result of verifying the expression of these genes was illustrated in FIG. 5.

As illustrated in FIG. 5, in mesenchymal stem cells (MSC) without an induction process, an expression level of OCT4 as a specific gene of the pluripotent stem cells was low, whereas in the pluripotent stem cells (EPN) induced by the method of the present invention, these specific genes were significantly highly expressed. The SOX2 and the Nanog as the stem cell genes were significantly higher expressed in the induced pluripotent stem cells (EPN) than the mesenchymal stem cells (MSC) and the c-Myc as the non-specific gene was lower expressed in the cells (EPN) with the induction process than the cells (MSC) without the induction process.

### Experimental Example 4: Differentiation into Ectodermal Cells (Nerve Cells)

In order to induce the differentiation to nerve cells, the cells were cultured in an incubator under a condition of humidity 95%, 37° C., and 5% CO2 by using a dedifferentiation medium STC-F002 according to the present invention, pluripotent stem cells were induced from the mesenchymal stem cells, cultured in a nerve cell differentiation solution of DMEM F-12, 2% B-27 supplement, 2 mM of L-glutamin, 30 ng/ml of EGF, and 25 ng/ml of bFGF for 5 days, and then cultured in a medium consisting of 2% fatal calf serum (FCS), 25 ng/ml of bFGF, and 25 ng/ml of a brain derived neurotrophic factor (BDNF) for 7 days. For verifying the differentiation into the nerve cells, a nestin protein was verified through immunohistochemical straining, and as a result, as illustrated in FIG. 6, it was verified that the cells were stained with green fluorescence and showed a positive reaction to be expected as pluripotent stem cells could be differentiated into ectodermal nerve cells.

### Experimental Example 5: Differentiation into Endoderm Cells (Liver Cells)

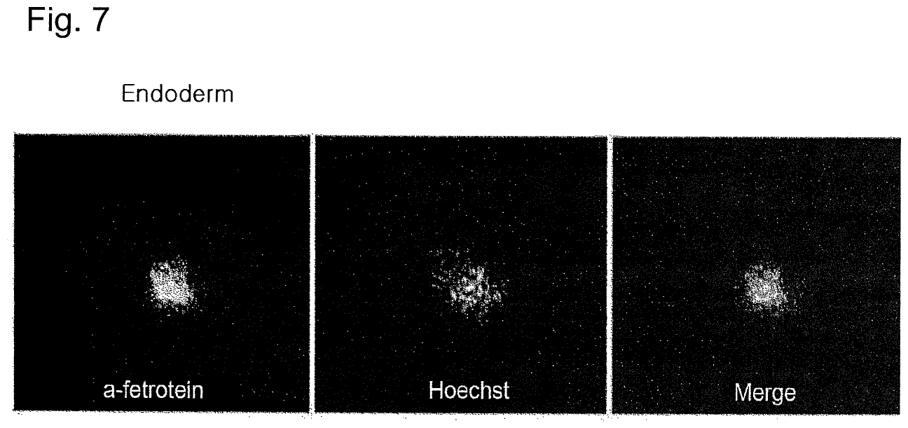

In order to induce the differentiation into liver cells, the cells were cultured in an incubator under the condition of humidity 95%, 37° C., and 5% CO2 by using a dedifferentiation medium STC-F002 according to the present invention, pluripotent stem cells were induced from the mesenchymal stem cells, and then, the induced cells were cultured in a liver cell differentiation solution of DMEM F-12, 20 nM dexamethason, 5.5 μg/ml of transferring, 7 ng/ml of sodium selenite, 100 ng/ml of HGF, 50 ng/ml of FGF, and 10 μg/ml of insulin for 3 weeks. For verifying the differentiation into the liver cells, the cells were verified through a-fetrotein immunohistochemical straining, and as a result, as illustrated in FIG. 7, it was verified that the cells were stained with green fluorescence and showed a positive reaction to be expected as pluripotent stem cells could be differentiated into liver cells as endoderm cells.

### Experimental Example 6: Differentiation into Mesodermal Cells (Cartilage and Osteoblast)

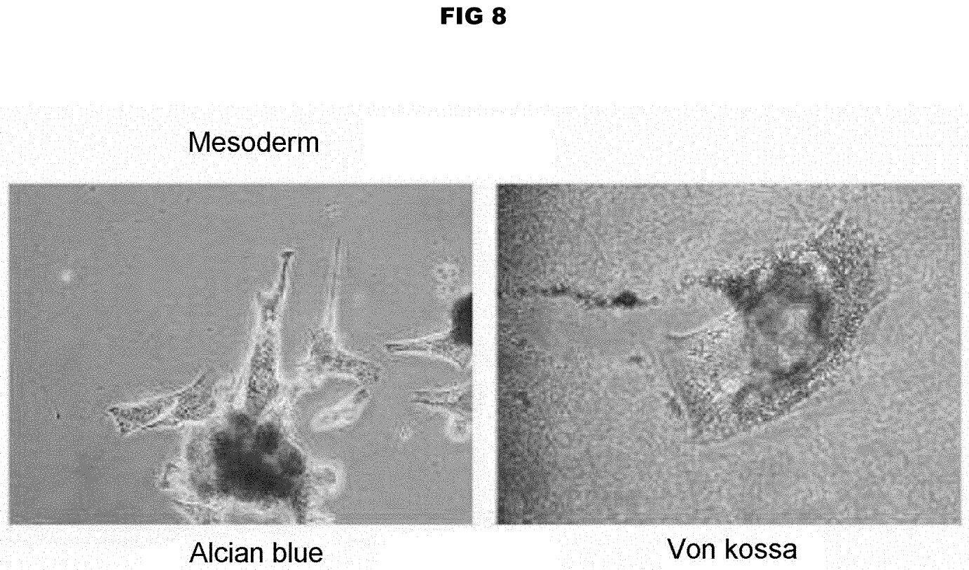

In order to induce the differentiation into cartilage cells, the cells were cultured in an incubator under the condition of humidity 95%, 37° C., and 5% CO2 by using a dedifferentiation medium STC-F002 according to the present invention to induce pluripotent stem cells from the mesenchymal stem cells, and then, the differentiated cells were cultured in a cartilage cell differentiation solution of DMEM F-12, 0.1 uM dexamethason, 50 μg/ml of Acetylsalicylic Acid (AsA), 100 μg/ml of sodium pyruvate, 40 μg/ml of proline, 10 ng/ml of TGF-.beta.1, 5% Insulin-Transferrin-Selenium (ITS; 6.25 μg/ml of insulin, 6.25 μg/ml of transferring, and 6.25 ng/ml of selenius acid), 1.25 mg/ml of bovine serum albumin, and 5.35 mg/ml of linoleic acid for 2 weeks. For verifying the differentiation into the cartilage cells, the cells were verified through Alcian blue histochemical straining, and as a result, as illustrated in FIG. 8, it was verified that the cells showed an Alcian blue positive reaction to be expected as pluripotent stem cells could be differentiated into the cartilage cells as the mesodermal cells.

Meanwhile, in order to induce the differentiation into osteoblasts, the cells were cultured in an incubator under the condition of humidity 95%, 37° C., and 5% CO2 by using a dedifferentiation medium STC-F002 according to the present invention, pluripotent stem cells were induced from the mesenchymal stem cells, and then, the induced cells were cultured in a osteoblast differentiation solution of DMEM F-12, 2 uM dexamethasone, 10 mM.beta.-glycerol phosphate, 0.3 mM ascorbic acid, and 1 uM bone morphogenic protein (BMP) for 2 weeks. For verifying the differentiation into the osteoblasts, the cells were verified through Von kossa histochemical straining, and as a result, as illustrated in FIG. 8, it was verified that the cells showed a Von kossa positive reaction to be expected as pluripotent stem cells could be differentiated into the osteoblasts as the mesodermal cells.

The invention has been described in detail with reference to preferred embodiments thereof. However, it will be appreciated by those skilled in the art that changes may be made in these embodiments without departing from the principles and spirit of the invention. Therefore, the scope of which is defined in the appended claims and their equivalents.

[Accession Number]

Depository institution name: Korean Cell Line Research Foundation

Accession Number: KCLRF-BP-00318

Accession date: May 30, 2014

What is claimed is:

1. An induced pluripotent stem cell line EPN-1 (deposit number: KCLRF-BP-00318) dedifferentiated by culturing mesenchymal stem cells in a dedifferentiation medium containing an Ecklonia cava extract.

2. The induced pluripotent stem cell line EPN-1 (deposit number: KCLRF-BP-00318) of claim 1, wherein the dedifferentiation medium includes 1 to 400 μg/ml of an Ecklonia cava extract based on the medium composition.

3. The induced pluripotent stem cell line EPN-1 (deposit number: KCLRF-BP-00318) of claim 1, wherein the induced pluripotent stem cell line exhibits a positive response in a straining reaction for Oct-4, SOX-2, or stage-specific embryonic antigen (SSEA-4).

4. The induced pluripotent stem cell line EPN-1 (deposit number: KCLRF-BP-00318) of claim 1, wherein the pluripotent stem cell line has ability capable of being naturally differentiated into ectodermal cells, endodermal cells, and mesodermal cells as embryo analogues.

5. A cell therapeutic composition including the induced pluripotent stem cell line (deposit number: KCLRF-BP-00318) of claim 1.

6. A method for treating a arthritis, neurological disorders, endocrine disorders or liver diseases, comprising the step of treating a subject with composition comprising an induced pluripotent stem cell line EPN-1 (deposit number: KCLRF-BP-00318).
